(19) United States
(12) Patent Application Publication
(10) Pub. No.: US 2022/0098169 A1
(43) Pub. Date: Mar. 31, 2022

(54) ANTIVIRAL AGENTS

(71) Applicant: TaiGen Biotechnology Co., Ltd., Taipei (TW)

(72) Inventors: Tai-Wei Ly, San Diego, CA (US); Shih-Chieh Chuang, New Taipei City (TW); Jhe-Ruei Guo, New Taipei City (TW); Chang-Pin Huang, New Taipei City (TW)

(21) Appl. No.: 17/391,081

(22) Filed: Aug. 2, 2021

Related U.S. Application Data
(60) Provisional application No. 63/083,146, filed on Sep. 25, 2020.

(51) Int. Cl. C07D 401/12

(52) U.S. Cl. CPC C07D 401/12

(57) ABSTRACT

A compound of Formula (I) below, or a pharmaceutically acceptable salt, stereoisomer, solvate, or prodrug thereof:

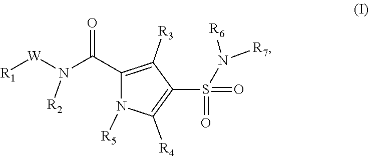

in which R₁-R₇, and W are defined as in the SUMMARY section. Further disclosed are a method of using the above-described compound, salt, stereoisomer, enantiomer, solvate, or prodrug for treating or preventing viral infectious diseases and a pharmaceutical composition containing same.

### TECHNICAL FIELD

This present disclosure is related to a phenyl-carboxamide derivative, a pharmaceutical composition thereof and the use of same in the treatment of viral infections, particularly hepatitis B virus infection.

### BACKGROUND

The hepatitis B virus (HBV) causes liver inflammation and injury that over several decades can further lead to serious complications, including cirrhosis and hepatocellular carcinoma. Hepatitis B is a significant public health threat, with over 250 million people living with hepatitis B worldwide. Nearly one million people die each year from hepatitis B and related diseases.

HBV is an enveloped DNA virus with an icosahedral core. The protein shell of the core, the capsid, is a self-assembling complex of 120 core protein homodimer. The correct assembly of the core proteins into a structurally and functionally relevant form is a key step for biological process to be carried out successfully. The capsid encloses the HBV DNA and a DNA polymerase that has reverse transcriptase activity. HBV replication is highly dependent on the accurate assembly of the capsid, which is also associated with the covalently closed circular DNA (cccDNA) reservoir for persistent infection. In addition to capsid assembly, core protein also has multiple roles in HBV lifecycle, making it an attractive drug target.

Interferons (IFNs) & nucleos(t)ide analogues (NAs) are currently available drugs for treatment of chronic HBV infection. However, the currently available treatments are suboptimal. There is a need in the art for the identification of novel compounds that can be used to treat and/or prevent HBV infection in a subject.

### SUMMARY

The present disclosure relates to a phenyl-carboxamide derivative as a class of virus inhibitors. Unexpectedly, these compounds showed effective anti-HBV activity.

Provided herein is a compound of Formula (I) below, or a pharmaceutically acceptable salt, or stereoisomer, solvate, or prodrug thereof:

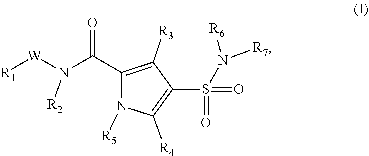

wherein, R₁ is C₁₋₆ alkyl, C₂₋₆ alkenyl, C₁₋₆ alkoxy, C₃₋₁₂ carbocyclyl, C₃₋₁₂ heterocyclyl, aryl, or C₅₋₁₀ heteroaryl, wherein said C₁₋₆ alkyl, C₂₋₆ alkenyl, C₁₋₆ alkoxy, C₃₋₁₂ carbocyclyl, C₃₋₁₂ heterocyclyl, aryl, and C₅₋₁₀ heteroaryl is each independently optionally substituted with 1 to 4 substituents each independently selected from hydrogen, halogen, NH₂, OH, CN, C₁₋₆ alkyl, C₂₋₆ alkenyl, C₁₋₆ alkoxy, C₁₋₆ haloalkyl, C₁₋₆ haloalkoxy, C₃₋₆ carbocyclyl, C₃₋₆ heterocyclyl, aryl, and C₅₋₆ heteroaryl;

W is a bond, C₁₋₆ alkyl, or C₂₋₆ alkenyl;

R₂ is hydrogen, C₁₋₆ alkyl, or C₁₋₆ haloalkyl;

R₃ is hydrogen, halogen, CN, C₁₋₆ alkyl or C₁₋₆ haloalkyl;

R₄ is hydrogen, halogen, CN, C₁₋₆ alkyl or C₁₋₆ haloalkyl;

R₅ is C₁₋₆ alkyl optionally substituted with 1 to 3 deuteriums;

R₆ is hydrogen, C₁₋₆ alkyl, or C₁₋₆ haloalkyl;

R₇ is C₃₋₁₂ heterocyclyl, aryl, C₅₋₁₀ heteroaryl, C₁₋₆ alkyl-C₃₋₁₂ heterocyclyl, C₁₋₆ alkyl-aryl, or C₁₋₆ alkyl-C₅₋₁₀ heteroaryl wherein said C₃₋₁₂ heterocyclyl, aryl, C₅₋₁₀ heteroaryl, C₁₋₆ alkyl-C₃₋₁₂ heterocyclyl, C₁₋₆ alkyl-aryl, or C₁₋₆ alkyl-C₅₋₁₀ heteroaryl is each independently optionally substituted with one or more substituents each independently selected from hydrogen, halogen, OH, CN, C₁₋₆ alkyl, C₂₋₆ alkenyl, C₁₋₆ alkoxy, C₁₋₆ haloalkyl, C₃₋₁₂ heterocyclyl, phosphate ester, C₁₋₆ alkyl-R₈, C₂₋₆ alkenyl-R₈, C₁₋₆ alkoxy-R₈, —NHS(O)₂—R₈, —S(O)₂—N(R₈)₂, —NHS(O)₂—N(R₈)₂, NH—C(═O)—N(R₈)₂, —C(═O)—C(═O)—R₈, NH—C(═O)—C(═O)—R₈, —C(═O)—C(═O)—N(R₈)₂, or NH—C(═O)—C(═O)—N(R₈)₂, wherein said C₁₋₆ alkyl, C₂₋₆ alkenyl, C₁₋₆ alkoxy, C₁₋₆ haloalkyl, C₃₋₁₂ heterocyclyl, phosphate ester, C₁₋₆ alkyl-R₈, C₂₋₆ alkenyl-R₈, C₂₋₆ alkoxy-R₈, —NHS(O)₂—R₈, —S(O)₂—N(R₈)₂, —NHS(O)₂—N(R₈)₂, NH—C(═O)—N(R₈)₂, —C(═O)—C(═O)—R₈, NH—C(═O)—C(═O)—R₈, —C(═O)—C(═O)—N(R₈)₂, or NH—C(═O)—C(═O)—N(R₈)₂ is each independently optionally substituted with one or more substituents each independently selected from hydrogen, halogen, OH, CN, oxo, C₁₋₆ alkyl, or C₁₋₆ haloalkyl;

R₈ is hydrogen, halogen, OH, CN, C₁₋₆ alkyl, C₂₋₆ alkenyl, C₁₋₆ alkoxy, C₃₋₁₂ carbocyclyl, C₃₋₁₂ heterocyclyl, aryl, or C₅₋₁₄ heteroaryl, wherein said C₁₋₆ alkyl, C₂₋₆ alkenyl, C₁₋₆ alkoxy, C₃₋₁₂ carbocyclyl, C₃₋₁₂ heterocyclyl, aryl, or C₅₋₁₄ heteroaryl is each independently optionally substituted with one or more halogen, OH, CN, C₁₋₆ alkyl, C₂₋₆ alkenyl, C₁₋₆ haloalkyl, or C₁₋₆ alkoxy.

Also provided herein are pharmaceutical compositions comprising a compound disclosed herein, e.g., a compound of Formula (I), including a stereoisomer, or enantiomer thereof; or a pharmaceutically acceptable salt, stereoisomer, solvate, or prodrug thereof; and one or more pharmaceutically acceptable carriers or excipients. The pharmaceutical composition can be used for treating viral infections, particularly HBV infection or diseases associated with HBV.

Further provided herein is a method of treating, preventing, or ameliorating viral infection, or one or more symptoms of a virus-mediated disorder, disease, or condition in a subject, comprising administering to the subject a therapeutically effective amount of a compound disclosed herein, e.g., a compound of Formula (I), including a stereoisomer, or enantiomer thereof; or a pharmaceutically acceptable salt, stereoisomer, solvate, or prodrug thereof, wherein said virus includes but are not limited to HBV, adenovirus, HIV, influenza virus, herpes virus, human papilloma virus. In related embodiments, the method may further comprise administering a compound disclosed herein, e.g., a compound of

Formula (I), including a stereoisomer, or enantiomer thereof; or a pharmaceutically acceptable salt, stereoisomer, solvate, or prodrug thereof, in combination with one or more additional therapeutic agents, wherein a compound disclosed herein and one or more additional therapeutic agents are administered either together in a single formulation, or administered separately in different formulations, and wherein the administration of the compound disclosed herein and the additional therapeutic agents is done concomitantly, or in series.

Additionally provided herein is a method of preparing a compound disclosed herein, e.g., a compound of Formula (I), including a stereoisomer, or enantiomer variant thereof; or a pharmaceutically acceptable salt, stereoisomer, solvate, or prodrug thereof.

### DETAILED DESCRIPTION

To facilitate understanding of the disclosure set forth herein, a number of terms are defined below.

Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs.

The term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, ±0.5% and ±0.1% from the specified value, as such variations are appropriate to perform the disclosed methods.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “patient”, “individual” or “subject” refers to a human or a non-human mammal. Preferably, the patient, individual, or subject is human.

The term “therapeutically effective amount” refers to the amount of an active compound is sufficient to prevent development of, or alleviate to some extent, one or more of the symptoms of the disorder, disease, or condition being treated.

The term “pharmaceutically acceptable carrier” or “pharmaceutically acceptable excipient” refers to a pharmaceutically-acceptable material, composition, or vehicle, such as a liquid or solid filler, diluents, solvent, or encapsulating material, which does not abrogate the biological activity or properties of the compound, and is relatively non-toxic, i.e., the material may be administered to an individual without causing undesirable biological effect or interacting in a deleterious manner with any of the components of the composition in which it is contained. See, Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “one or more” refers to either one or a number above one (e.g. 2, 3, 4, 5, 6, or 7).

The term “halo” or “halogen” (alone or as part of another substituent) refers to a fluorine, chlorine, bromine, or iodine atom.

The term “C₁₋₆ alkyl” (alone or in combination with another term) refers to a straight- or branched-chain saturated hydrocarbyl substituent containing 1 to 6 (e.g., 1 to 4) carbon atoms. Examples of C₁₋₆ alkyl include methyl, ethyl, n-propyl, isopropyl, n-butyl, isobutyl, sec-butyl, tert-butyl, n-pentyl, and the like.

The term “C₂₋₆ alkenyl” (alone or in combination with another term) refers to a straight- or branched-chain hydrocarbyl substituent containing 2 to 6 (e.g., 2 to 4) carbon atoms and one or more double bonds. Examples of C₂₋₆ alkenyl include vinyl, allyl, propenyl, isopropenyl, butenyl, isobutenyl, prenyl, butadienyl, pentenyl, isopentenyl, pentadienyl, and the like.

The term “C₂₋₆ alkynyl” (alone or in combination with another term) refers to a straight- or branched-chain hydrocarbyl substituent containing 2 to 6 (e.g., 2 to 4, 2 to 3) carbon atoms and one or more triple bonds. Examples of C₂₋₆ alkynyl include ethynyl, propynyl, butynyl, pentynyl, and the like.

The term “C₁₋₆ alkoxy” (alone or in combination with another term) refers to the group —OR′ wherein R′ is C₁₋₆ alkyl. Examples of C₁₋₆ alkoxy include methoxy, ethoxy, n-propoxy, isopropoxy, n-butoxy, iso-butoxy, sec-butoxy, and tert-butoxy.

The term “haloalkyl” refers to an alkyl group substituted by one or more halogens, wherein the alkyl is as defined above.

The term “haloalkoxy” refers to an alkoxy group substituted by one or more halogens, wherein the alkoxy is as defined above.

The term “cycloalkyl” refers to a cyclic monovalent hydrocarbon radical. In one embodiment, cycloalkyl groups may be saturated or unsaturated but non-aromatic. In certain embodiments, the cycloalkyl has from 3 to 12 (C₃₋₁₂), 3 to 6 (C₃₋₆), from 4 to 6 (C₄₋₆), or from 5 to 6 (C₅₋₆) carbon atoms. Examples of cycloalkyl groups include, but are not limited to, cyclopropyl, cyclobutyl, cyclopentyl, cyclopentenyl, and cyclohexyl.

The term “carbocyclyl” refers to a saturated (i.e., “cycloalkyl”) or partly unsaturated (i.e., “cycloalkenyl”) monocyclic or bicyclic (fused, bridged, or spiro) ring containing from 3 to 12 (C₃₋₁₂) ring atoms. In certain embodiments, the carbocyclyl has from 3 to 10 (C₃₋₁₀), from 3 to 8 (C₃₋₈), from 4 to 8 (C₄₋₈), from 3 to 6 (C₃₋₆), from 4 to 6 (C₄₋₆), or from 5 to 6 (C₅₋₆) ring atoms. Examples of such carbocyclyl include, but are not limited to, cyclopropyl, cyclobutyl, cyclopentyl, cyclopentenyl, cyclohexyl, cyclohexenyl, cycloheptyl, norbornyl, and norbornenyl.

The term “heterocyclyl” refers to a saturated (i.e., “heterocycloalkyl”) or partly unsaturated (i.e., “heterocycloalkenyl” monocyclic or bicyclic (fused, bridged, or spiro) ring containing from 3 to 12 (C₃₋₁₂) ring atoms which can comprise one, two or three heteroatoms selected from O, S, P, and N. In certain embodiments, the heterocyclyl has from 3 to 10 (C₃₋₁₀), from 3 to 8 (C₃₋₈), from 4 to 8 (C₄₋₈), from 3 to 6 (C₃₋₆), from 4 to 6 (C₄₋₆), or from 5 to 6 (C₅₋₆) ring atoms. The heterocyclyl may be attached to the main structure at any heteroatom or carbon atom which results in the creation of a stable compound. Examples of such heterocyclyl include, but are not limited to, azetidinyl, pyrrolidinyl, piperidinyl, piperazinyl, morpholinyl, oxetanyl, tetrahydropyranyl, tetrahydrothiopyranyl, dioxaphospholane, and tetrahydrofuryl.

The term “aryl” refers to a monovalent monocyclic aromatic group and/or monovalent polycyclic aromatic group that contain at least one aromatic carbon ring. In certain embodiments, the aryl has from 6 to 20 (C₆₋₂₀), from 6 to 14 (C₆₋₁₄), or from 6 to 10 (C₆₋₁₀) ring atoms. Examples of aryl groups include, but are not limited to, phenyl, naphthyl, fluorenyl, azulenyl, anthryl, phenanthryl, pyrenyl, biphenyl, and terphenyl. Aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl).

The term “heteroaryl” refers to a monovalent monocyclic aromatic group or monovalent polycyclic aromatic group that contain at least one aromatic ring, wherein at least one aromatic ring can contain one, two, three or four heteroatoms independently selected from O, S, P, and N in the ring. Heteroaryl groups are bonded to the rest of a molecule through the aromatic ring. Each ring of a heteroaryl group can contain one or two O atoms, one or two S atoms, and/or one to four N atoms, provided that the total number of heteroatoms in each ring is four or less and each ring contains at least one carbon atom. In certain embodiments, the heteroaryl has from 5 to 20 (C₅₋₂₀), from 5 to 14 (C₅₋₁₄), or from 5 to 10 (C₅₋₁₀) ring atoms. Examples of monocyclic heteroaryl groups include, but are not limited to, furanyl, imidazolyl, isothiazolyl, isoxazolyl, oxadiazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl.

The term “phosphate ester” refers to an ester formed from phosphoric acid. Phosphate ester can be acyclic or cyclic. The acyclic phosphate ester refers to the PO₃ central portion of an organophosphate having up to three side chains. The side chain including but not limited to hydrogen or C₁₋₆ alkyl. The cyclic phosphate ester refers to the group

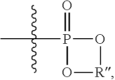

wherein R″ represents a unsubstituted or substituted C₁₋₆ alkyl. Examples of the cyclic phosphate ester include but not limited to 1,3,2-dioxaphosphetane-2-oxide, 1,3,2-dioxaphospholane-2-oxide, 1,3,2-dioxaphosphinane-2-oxide, and the like.

The term “protecting group” refers to a chemical group known in the art that are readily introduced on to and removed from a nitrogen atom or an oxygen atom. Examples of nitrogen protecting group includes but is not limited to alkoxycarbonyls, acyls, alkyls; for example tert-butyloxycarbonyl, benzyloxycarbonyl, fluorene-methoxycarbonyl, allylloxycarbonyl, phthalyl, benzyl, para-methoxybenzyl, triphenylmethyl or the like. It can be appropriately selected and manipulated by those skilled in the art with reference to the conventional textbook in the art, such as Greene's Protective Groups in Organic Synthesis (4^{th} edition).

The term “solvate” refers to a complex formed between an active compound and a pharmaceutically acceptable solvent. Examples of pharmaceutically acceptable solvents include water, ethanol, isopropanol, ethyl acetate, acetic acid, and ethanolamine.

The disclosure provides herein relate to a compound of Formula (I), or a pharmaceutically acceptable salt, stereoisomer, solvate, or prodrug thereof:

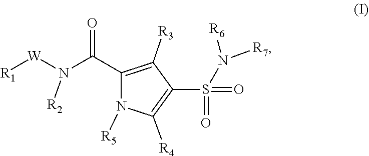

wherein, R₁ is C₁₋₆ alkyl, C₂₋₆ alkenyl, C₁₋₆ alkoxy, C₃₋₁₂ carbocyclyl, C₃₋₁₂ heterocyclyl, aryl, or C₅₋₁₀ heteroaryl, wherein said C₁₋₆ alkyl, C₂₋₆ alkenyl, C₁₋₆ alkoxy, C₃₋₁₂ carbocyclyl, C₃₋₁₂ heterocyclyl, aryl, and C₅₋₁₀ heteroaryl is each independently optionally substituted with 1 to 4 substituents each independently selected from hydrogen, halogen, NH₂, OH, CN, C₁₋₆ alkyl, C₂₋₆ alkenyl, C₁₋₆ alkoxy, C₁₋₆ haloalkyl, C₁₋₆ haloalkoxy, C₃₋₆ carbocyclyl, C₃₋₆ heterocyclyl, aryl, and C₅₋₆ heteroaryl;

W is a bond, C₁₋₆ alkyl, or C₂₋₆ alkenyl;

R₂ is hydrogen, C₁₋₆ alkyl, or C₁₋₆ haloalkyl;

R₃ is hydrogen, halogen, CN, C₁₋₆ alkyl or C₁₋₆ haloalkyl;

R₄ is hydrogen, halogen, CN, C₁₋₆ alkyl or C₁₋₆ haloalkyl;

R₅ is C₁₋₆ alkyl optionally substituted with 1 to 3 deuteriums;

R₆ is hydrogen, C₁₋₆ alkyl, or C₁₋₆ haloalkyl;

R₇ is C₃₋₁₂ heterocyclyl, aryl, C₅₋₁₀ heteroaryl, C₁₋₆ alkyl-C₃₋₁₂ heterocyclyl, C₁₋₆ alkyl-aryl, or C₁₋₆ alkyl-C₅₋₁₀ heteroaryl wherein said C₃₋₁₂ heterocyclyl, aryl, C₅₋₁₀ heteroaryl, C₁₋₆ alkyl-C₃₋₁₂ heterocyclyl, C₁₋₆ alkyl-aryl, or C₁₋₆ alkyl-C₅₋₁₀ heteroaryl is each independently optionally substituted with one or more substituents each independently selected from hydrogen, halogen, OH, CN, C₁₋₆ alkyl, C₂₋₆ alkenyl, C₁₋₆ alkoxy, C₁₋₆ haloalkyl, C₃₋₁₂ heterocyclyl, phosphate ester, C₁₋₆ alkyl-R₈, C₂₋₆ alkenyl-R₈, C₁₋₆ alkoxy-R₈, —NHS(O)₂—R₈, —S(O)₂—N(R₈)₂, —NHS(O)₂—N(R₈)₂, NH—C(═O)—N(R₈)₂, —C(═O)—C(═O)—R₈, NH—C(═O)—C(═O)—R₈, —C(═O)—C(═O)—N(R₈)₂, or NH—C(═O)—C(═O)—N(R₈)₂, wherein said C₁₋₆ alkyl, C₂₋₆ alkenyl, C₁₋₆ alkoxy, C₁₋₆ haloalkyl, C₃₋₁₂ heterocyclyl, phosphate ester, C₁₋₆ alkyl-R₈, C₂₋₆ alkenyl-R₈, C₁₋₆ alkoxy-R₈, —NHS(O)₂—R₈, —S(O)₂—N(R₈)₂, —NHS(O)₂—N(R₈)₂, NH—C(═O)—N(R₈)₂, —C(═O)—C(═O)—R₈, NH—C(═O)—C(═O)—R₈, —C(═O)—C(═O)—N(R₈)₂, or NH—C(═O)—C(═O)—N(R₈)₂ is each independently optionally substituted with one or more substituents each independently selected from hydrogen, halogen, OH, CN, oxo, C₁₋₆ alkyl, or C₁₋₆ haloalkyl;

R₈ is hydrogen, halogen, OH, CN, C₁₋₆ alkyl, C₂₋₆ alkenyl, C₁₋₆ alkoxy, C₃₋₁₂ carbocyclyl, C₃₋₁₂ heterocyclyl, aryl, or C₅₋₁₄ heteroaryl, wherein said C₁₋₆ alkyl, C₂₋₆ alkenyl, C₁₋₆ alkoxy, C₃₋₁₂ carbocyclyl, C₃ heterocyclyl, aryl, or C₅₋₁₄ heteroaryl is each independently optionally substituted with one or more halogen, OH, CN, C₁₋₆ alkyl, C₂₋₆ alkenyl, C₁₋₆ haloalkyl, or C₁₋₆ alkoxy.

In one embodiment, provided herein is a compound of Formula (II), or a pharmaceutically acceptable salt, stereoisomer, solvate, or prodrug thereof:

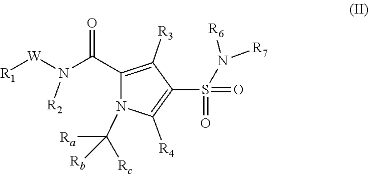

wherein

R₁ is C₁₋₆ alkyl, C₂₋₆ alkenyl, C₁₋₆ alkoxy, C₃₋₁₂ carbocyclyl, C₃₋₁₂ heterocyclyl, aryl, or C₅₋₁₀ heteroaryl, wherein said C₁₋₆ alkyl, C₂₋₆ alkenyl, C₁₋₆ alkoxy, C₃₋₁₂ carbocyclyl, C₃₋₁₂ heterocyclyl, aryl, and C₅₋₁₀ heteroaryl is each independently optionally substituted with 1 to 4 substituents each independently selected from hydrogen, halogen, NH₂, OH, CN, C₁₋₆ alkyl, C₂₋₆ alkenyl, C₁₋₆ alkoxy, C₁₋₆ haloalkyl, C₁₋₆ haloalkoxy, C₃₋₆ carbocyclyl, C₃₋₆ heterocyclyl, aryl, and C₅₋₆ heteroaryl;

W is a bond, C₁₋₆ alkyl, or C₂₋₆ alkenyl;

R₂ is hydrogen, C₁₋₆ alkyl, or C₁₋₆ haloalkyl;

R₃ is hydrogen, halogen, CN, C₁₋₆ alkyl or C₁₋₆ haloalkyl;

R₄ is hydrogen, halogen, CN, C₁₋₆ alkyl or C₁₋₆ haloalkyl;

R₆ is hydrogen, C₁₋₆ alkyl, or C₁₋₆ haloalkyl;

R₇ is C₃₋₁₂ heterocyclyl, aryl, C₅₋₁₀ heteroaryl, C₁₋₆ alkyl-C₃₋₁₂ heterocyclyl, C₁₋₆ alkyl-aryl, or C₁₋₆ alkyl-C₅₋₁₀ heteroaryl wherein said C₃₋₁₂ heterocyclyl, aryl, C₅₋₁₀ heteroaryl, C₁₋₆ alkyl-C₃₋₁₂ heterocyclyl, C₁₋₆ alkyl-aryl, or C₁₋₆ alkyl-C₅₋₁₀ heteroaryl is each independently optionally substituted with one or more substituents each independently selected from hydrogen, halogen, OH, CN, C₁₋₆ alkyl, C₂₋₆ alkenyl, C₁₋₆ alkoxy, C₁₋₆ haloalkyl, C₃₋₁₂ heterocyclyl, phosphate ester, C₁₋₆ alkyl-R₈, C₂₋₆ alkenyl-R₈, C₁₋₆ alkoxy-R₈, —NHS(O)₂—R₈, —S(O)₂—N(R₈)₂, —NHS(O)₂—N(R₈)2, NH-C(═O)—N(R₈)₂, —C(═O)—C(═O)—R₈, NH—C(═O)—C(═O)—R₈, —C(═O)—C(═O)—N(R₈)₂, or NH—C(═O)—C(═O)—N(R₈)₂, wherein said C₁₋₆ alkyl, C₂₋₆ alkenyl, C₁₋₆ alkoxy, C₁₋₆ haloalkyl, C₃₋₁₂ heterocyclyl, phosphate ester, C₁₋₆ alkyl-R₈, C₂₋₆ alkenyl-R₈, C₁₋₆ alkoxy-R₈, —NHS(O)₂—R₈, —S(O)₂—NR₈)₂, —NHS(O)₂—NR₈)₂, NH—C═O)—N(R₈)₂, —C(═O)—C(═O)—R₈, NH—C(═O)—C(═O)—R₈, —C(═O)—C(═O)—N(R₈)₂, or NH—C(═O)—C(═O)—N(R₈)₂ is each independently optionally substituted with one or more substituents each independently selected from hydrogen, halogen, OH, CN, oxo, C₁₋₆ alkyl, or C₁₋₆ haloalkyl;

R₈ is hydrogen, halogen, OH, CN, C₁₋₆ alkyl, C₂₋₆ alkenyl, C₁₋₆ alkoxy, C₃₋₁₂ carbocyclyl, C₃₋₁₂ heterocyclyl, aryl, or C₅₋₁₄ heteroaryl, wherein said C₁₋₆ alkyl, C₂₋₆ alkenyl, C₁₆ alkoxy, C₃₋₁₂ carbocyclyl, C₃₋₁₂ heterocyclyl, aryl, or C₅₋₁₄ heteroaryl is each independently optionally substituted with one or more halogen, OH, CN, C₁₋₆ alkyl, C₂₋₆ alkenyl, C₁₋₆ haloalkyl, or C₁₋₆ alkoxy;

each of Rₐ, R_{b} or R_{c}, independently, is hydrogen or deuterium.

In yet another embodiment, provided herein is a compound of Formula (III), or a pharmaceutically acceptable salt, stereoisomer, solvate, or prodrug thereof:

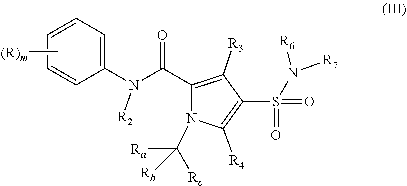

wherein

R is hydrogen, halogen, NH₂, OH, CN, C₁₋₆ alkyl, C₂₋₆ alkenyl, C₁₋₆ alkoxy, C₁₋₆ haloalkyl, C₁₋₆ haloalkoxy, C₃₋₆ carbocyclyl, C₃₋₆ heterocyclyl, aryl, or C₅₋₆ heteroaryl;

m is an integer of 1 to 4;

R₂ is hydrogen, C₁₋₆ alkyl, or C₁₋₆ haloalkyl;

R₃ is hydrogen, halogen, CN, C₁₋₆ alkyl or C₁₋₆ haloalkyl;

R₄ is hydrogen, halogen, CN, C₁₋₆ alkyl or C₁₋₆ haloalkyl;

R₆ is hydrogen, C₁₋₆ alkyl, or C₁₋₆ haloalkyl;

R₇ is C₃₋₁₂ heterocyclyl, aryl, C₅₋₁₀ heteroaryl, C₁₋₆ alkyl-C₃₋₁₂ heterocyclyl, C₁₋₆ alkyl-aryl, or C₁₋₆ alkyl-C₅₋₁₀ heteroaryl wherein said C₃₋₁₂ heterocyclyl, aryl, C₅₋₁₀ heteroaryl, C₁₋₆ alkyl-C₃₋₁₂ heterocyclyl, C₁₋₆ alkyl-aryl, or C₁₋₆ alkyl-C₅₋₁₀ heteroaryl is each independently optionally substituted with one or more substituents each independently selected from hydrogen, halogen, OH, CN, C₁₋₆ alkyl, C₂₋₆ alkenyl, C₁₋₆ alkoxy, C₁₋₆ haloalkyl, C₃₋₁₂ heterocyclyl, phosphate ester, C6₁₋₆ alkyl-R₈, C₂₋₆ alkenyl-R₈, C₁₋₆ alkoxy-R₈, —NHS(O)₂—R₈, —S(O)₂ —N(R₈)₂, —NHS(O)₂—N(R₈)2, NH—C(═O)—N(R₈)₂, —C(═O)—C(═O)—R₈, NH—C(═O)—C(═O)—R₈, —C(═O)—C(═O)—N(R₈)₂, or NH—C(═O)—C(═O)—N(R₈)₂, wherein said C₁₋₆ alkyl, C₂₋₆ alkenyl, C₁₋₆ alkoxy, C₁₋₆ haloalkyl, C₃₋₁₂ heterocyclyl, phosphate ester, C₁₋₆ alkyl-R₈, C₂₋₆ alkenyl-R₈, C₁₋₆ alkoxy-R₈, —NHS(O)₂—R₈, —S(O)₂—N(R₈)₂, —NHS(O)₂—N(R₈)₂, NH—C(═O)—N(R₈)₂, —C(═O)—C(═O)—R₈, NH—C(═O)—C(═O)—R₈, —C(═O)—C(═O)—N(R₈)₂, or NH—C(═O)—C(═O)—N(R₈)₂ is each independently optionally substituted with one or more substituents each independently selected from hydrogen, halogen, OH, CN, oxo, C₁₋₆ alkyl, or C₁₋₆ haloalkyl;

R₈ is hydrogen, halogen, OH, CN, C₁₋₆ alkyl, C₂₋₆ alkenyl, C₁₋₆ alkoxy, C₃₋₁₂ carbocyclyl, C₃₋₁₂ heterocyclyl, aryl, or C₅₋₁₄ heteroaryl, wherein said C₁₋₆ alkyl, C₂₋₆ alkenyl, C₁₋₆ alkoxy, C₃₋₁₂ carbocyclyl, C₃₋₁₂ heterocyclyl, aryl, or C₅₋₁₄ heteroaryl is each independently optionally substituted with one or more halogen, OH, CN, C₁₋₆ alkyl, C₂₋₆ alkenyl, C₁₋₆ haloalkyl, or C₁₋₆ alkoxy;

each of Rₐ, R_{b} or R_{c}, independently, is hydrogen or deuterium.

In yet another embodiment, provided herein is a compound of Formula (IV), or a pharmaceutically acceptable salt, stereoisomer, solvate, or prodrug thereof:

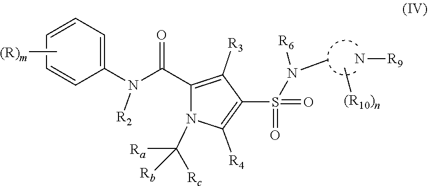

wherein

R is hydrogen, halogen, NH₂, OH, CN, C₁₋₆ alkyl, C₂₋₆ alkenyl, C₁₋₆ alkoxy, C₁₋₆ haloalkyl, C₁₋₆ haloalkoxy, C₃₋₆ carbocyclyl, C₃₋₆ heterocyclyl, aryl, or C₅₋₆ heteroaryl;

m is an integer of 1 to 4;

R₂ is hydrogen, C₁₋₆ alkyl, or C₁₋₆ haloalkyl;

R₃ is hydrogen, halogen, CN, C₁₋₆ alkyl or C₁₋₆ haloalkyl;

R₄ is hydrogen, halogen, CN, C₁₋₆ alkyl or C₁₋₆ haloalkyl;

R₆ is hydrogen, C₁₋₆ alkyl, or C₁₋₆ haloalkyl;

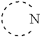

is a C₃₋₁₂ heterocyclyl containing one or two nitrogen atoms;

R₉ is hydrogen, C₃₋₁₂ heterocyclyl, phosphate ester, C₁₋₆ alkyl-R₈, C₂₋₆ alkenyl-R₈, C₁₋₆ alkoxy-R₈, —NHS(O)₂—R₈, —S(O)₂—N(R₈)₂, —NHS(O)₂—N(R₈)₂, NH—C(═O)—N(R₈)₂, —C(═O)—C(═O)—R₈, NH—C(═O)—C(═O)—R₈, —C(═O)—C(═O)—N(R₈)₂, or NH—C(═O)—C(═O)—N(R₈)₂, wherein said C₃₋₁₂ heterocyclyl, phosphate ester, C₁₋₆ alkyl-R₈, C₂₋₆ alkenyl-R₈, C₁₋₆ alkoxy-R₈, —NHS(O)₂—R₈, —S(O)₂—N(R₈)₂, —NHS(O)₂—N(R₈)₂, NH—C(═O)—N(R₈)₂, —C(═O)—C(═O)—R₈, NH—C(═O)—C(═O)—R₈, —C(═O)—C(═O)—N(R₈)₂, or NH—C(═O)—C(═O)—N(R₈)₂ is each independently optionally substituted with one or more substituents each independently selected from hydrogen, halogen, OH, CN, oxo, C₁₋₆ alkyl, or C₁₋₆ haloalkyl;

R₁₀ is hydrogen, halogen, OH, CN, C₁₋₆ alkyl, C₂₋₆ alkenyl, C₁₋₆ alkoxy, or C₁₋₆ haloalkyl;

n is an integer of 1 to 4;

R₈ is hydrogen, halogen, OH, CN, C₁₋₆ alkyl, C₂₋₆ alkenyl, C₁₋₆ alkoxy, C₃₋₁₂ carbocyclyl, C₃₋₁₂ heterocyclyl, aryl, or C₅₋₁₄ heteroaryl, wherein said C₁₋₆ alkyl, C₂₋₆ alkenyl, C₁₋₆ alkoxy, C₃₋₁₂ carbocyclyl, C₃₋₁₂ heterocyclyl, aryl, or C₅₋₁₄ heteroaryl is each independently optionally substituted with one or more halogen, OH, CN, C₁₋₆ alkyl, C₂₋₆ alkenyl, C₁₋₆ haloalkyl, or C₁₋₆ alkoxy;

each of Rₐ, R_{b} or R_{c}, independently, is hydrogen or deuterium.

The following embodiments are inclusive of definitions for Formula (I), (II) (III) and/or (IV).

In one embodiment, R₁ is aryl optionally substituted with 1 to 4 substituents each independently selected from hydrogen, halogen, NH₂, OH, CN, C₁₋₆ alkyl, C₂₋₆ alkenyl, C₁₋₆ alkoxy, C₁₋₆ haloalkyl, C₁₋₆ haloalkoxy, C₃₋₆ carbocyclyl, C₃₋₆ heterocyclyl, aryl, and C₅₋₆ heteroaryl. In another embodiment, R₁ is phenyl optionally substituted with 1 to 4 substituents each independently selected from hydrogen, halogen, CN, C₁₋₆ alkyl, C₂₋₆ alkenyl, C₁₋₆ alkoxy, C₁₋₆ haloalkyl, and C₁₋₆ haloalkoxy.

In one embodiment, W is a bond or C₁₋₆ alkyl. In another embodiment, W is a bond.

In one embodiment, R₂ is hydrogen or C₁₋₆ alkyl. In another embodiment, R₂ is hydrogen.

In one embodiment, R₃ is halogen, C₁₋₆ alkyl or C₁₋₆ haloalkyl. In another embodiment, R₃ is methyl, fluoro, chloro, or bromo. In yet another embodiment, R₃ is fluoro, chloro, or bromo.

In one embodiment, R₄ is halogen, C₁₋₆ alkyl or C₁₋₆ haloalkyl. In another embodiment, R₄ is fluoro, chloro, bromo, or methyl.

In one embodiment, R₅ is methyl optionally substituted with 1 to 3 deuteriums. In another embodiment, R₅ is methyl substituted with 1 to 3 deuteriums. In yet another embodiment, R₅ is CD₃.

In one embodiment, R₆ is hydrogen or C₁₋₆ alkyl. In another embodiment, R₆ is hydrogen or methyl. In yet another embodiment, R₆ is hydrogen.

In one embodiment, R₇ is C₃₋₁₂ heterocyclyl or C₁₋₆ alkyl-C₃₋₁₂ heterocyclyl, wherein said C₃₋₁₂ heterocyclyl or C₁₋₆ alkyl-C₃₋₁₂ heterocyclyl is each independently optionally substituted with one or more substituents each independently selected from hydrogen, halogen, OH, CN, C₃₋₁₂ heterocyclyl, phosphate ester, C₁₋₆ alkyl-R₈, C₂₋₆ alkenyl-R₈, C₁₋₆ alkoxy-R₈, —NHS(O)₂—R₈, —S(O)₂—N(R₈)₂, NH—C(═O)—N(R₈)₂, —C(═O)—C(═O)—R₈, NH—C(═O)—C(═O)—R₈, —C(═O)—C(═O)—N(R₈)₂, or NH—C(═O)—C(═O)—N(R₈)2. In another embodiment, R₇ is C₃₋₁₂ heterocyclyl containing one or two nitrogen atoms, wherein said C₃₋₁₂ heterocyclyl optionally substituted with one or more substituents each independently selected from hydrogen, halogen, OH, CN, C₃₋₁₂ heterocyclyl, phosphate ester, C₁₋₆ alkyl-R₈, C₂₋₆ alkenyl-R₈, C₁₋₆ alkoxy-R₈, —NHS(O)₂—R₈, —S(O)₂—N(R₈)₂, —NHS(O)₂—N(R₈)₂, NH—C(═O)—N(R₈)₂, —C(═O)—C(═O)—R₈, NH—C(═O)—C(═O)—R₈, —C(═O)—C(═O)—N(R₈)₂, or NH—C(═O)—C(═O)—N(R₈)₂. In yet another embodiment, R₇ is C₅₋₆ heterocyclyl containing one nitrogen atom, wherein said C₅₋₆ heterocyclyl optionally substituted with one or more substituents each independently selected from hydrogen, halogen, CN, C₃₋₁₂ heterocyclyl, phosphate ester, —S(O)₂—N(R₈)₂, or —C(═O)—C(═O)—N(R₈)₂.

In one embodiment, R₇ is

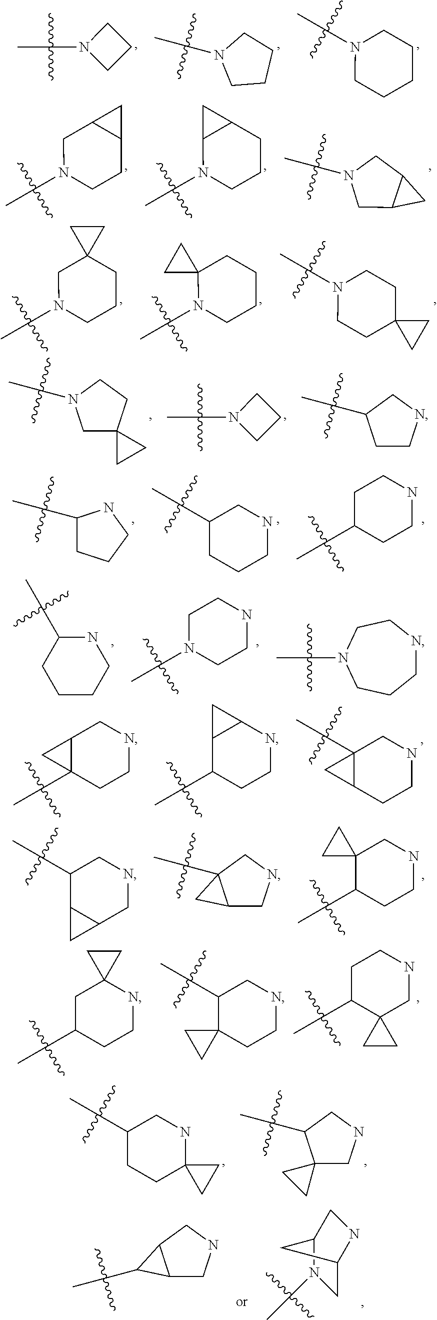

wherein each of the heterocyclyl moieties is optionally substituted with one or more substituents each independently selected from hydrogen, halogen, OH, CN, C₃₋₁₂ heterocyclyl, phosphate ester, C₁₋₆ alkyl-R₈, C₂₋₆ alkenyl-R₈, C₁₋₆ alkoxy-R₈, —NHS(O)₂—R₈, —S(O)₂—N(R₈)₂, —NHS(O)₂—N(R₈)₂, NH—C(═O)—N(R₈)₂, —C(═O)—C(═O)—R₈, NH—C(═O)—C(═O)—R₈, —C(═O)—C(═O)—N(R₈)₂, or NH—C(═O)—C(═O)—N(R₈)₂; In another embodiment, R₇ is

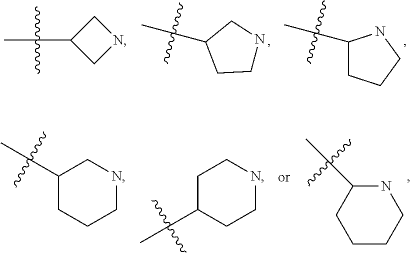

wherein each of the heterocyclyl moieties is optionally substituted with one or more substituents each independently selected from hydrogen, halogen, OH, CN, C₃₋₁₂ heterocyclyl, phosphate ester, C₁₋₆ alkyl-R₈, C₂₋₆ alkenyl-R₈, C₁₋₆ alkoxy-R₈, —NHS(O)₂—R₈, —S(O)₂—N(R₈)₂, —NHS(O)₂—N(R₈)₂, NH—C(═O)—N(R₈)₂, —C(═O)—C(═O)—R₈, NH—C(═O)—C(═O)—R₈, —C(═O)—C(═O)—N(R₈)₂, or NH—C(═O)'C(═O)—N(R₈)₂; In another embodiment, R₇ is

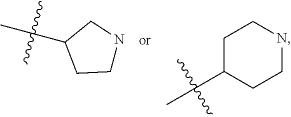

wherein each of the heterocyclyl moieties is optionally substituted with one or more substituents each independently selected from hydrogen, halogen, OH, CN, C₃₋₁₂ heterocyclyl, phosphate ester, C₁₋₆ alkyl-R₈, C₂₋₆ alkenyl-R₈, C₁₋₆ alkoxy-R₈, —NHS(O)₂—R₈, —S(O)₂—N(R₈)₂, —NHS(O)₂—N(R₈)₂, NH—C(═O)—N(R₈)₂, —C(═O)—C(═O)—R₈, NH—C(═O)—C(═O)⇒R₈, —C(═O)—C(═O)—N(R₈)₂, or NH—C(═O)—C(═O)—N(R₈)₂.

In one embodiment,

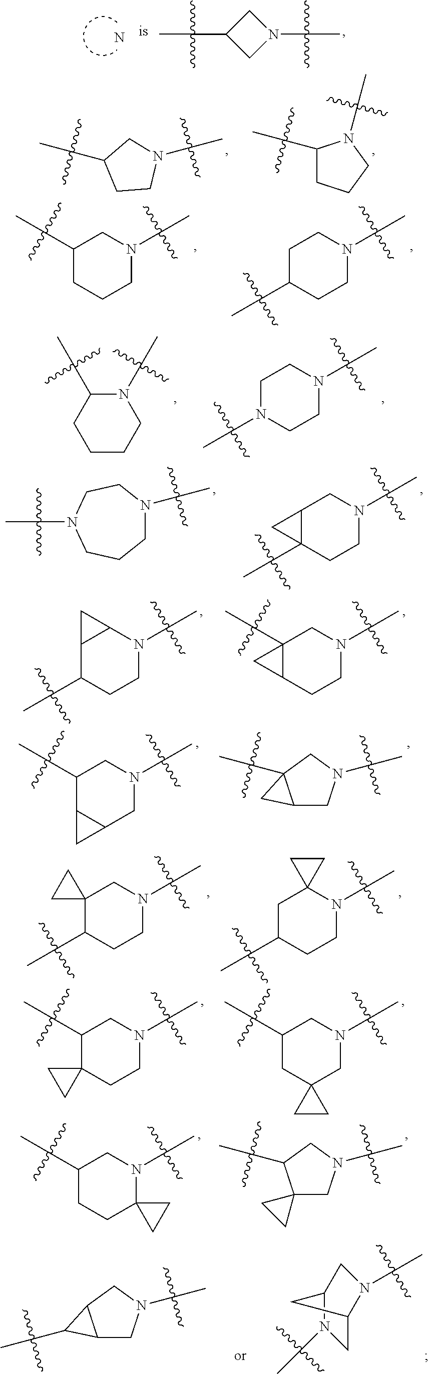

In another embodiment,

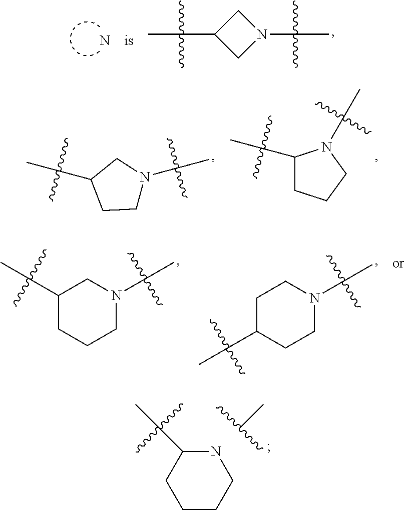

In yet another embodiment,

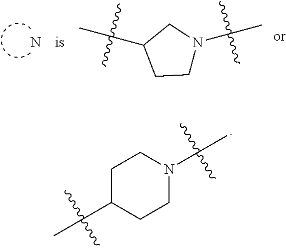

In one embodiment, R₈ is hydrogen, halogen, OH, CN, oxo, C₁₋₆ alkyl, C₂₋₆ alkenyl, or C₁₋₆ alkoxy, wherein said C₁₋₆ alkyl, C₂₋₆ alkenyl, or C₁₋₆ alkoxy is each independently optionally substituted with one or more halogen, OH, or CN.

In one embodiment, R₉ is hydrogen, C₃₋₆ heterocyclyl, phosphate ester, —NHS(O)₂—R₈, —S(O)₂—N(R₈)₂, —NHS(O)₂—N(R₈)₂, NH—C(═O)—N(R₈)₂, —C(═O)—C(═O)—R₈, NH—C(═O)—C(═O)—R₈, —C(═O)—C(═O)—N(R₈)₂, or NH—C(═O)—C(═O)—N(R₈)₂; In yet another embodiment, R₉ is hydrogen, phosphate ester, —S(O)₂—N(R₈)₂, or —C(═O)—C(═O)—N(R₈)₂.

In one embodiment, R is hydrogen, halogen, CN, C₁₋₆ alkyl, C₂₋₆ alkenyl, C₁₋₆ alkoxy, C₁₋₆ haloalkyl, or C₁₋₆haloalkoxy; In another embodiment, R is hydrogen, halogen, CN, or C₁₋₆ alkyl.

In one embodiment, each of Rₐ, R_{b} or R_{c}, independently, is hydrogen or deuterium, and at least one of Rₐ, R_{b} or R_{c} is deuterium.

In one embodiment, examples of the compound provided herein are shown as below:

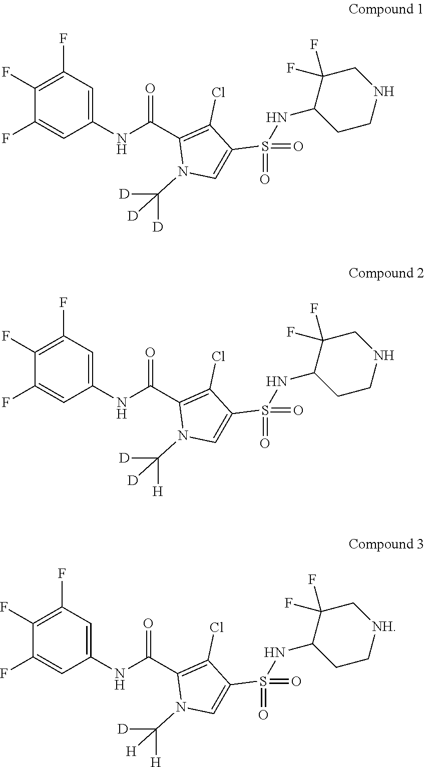 [SMILES: [2H]C([2H])([2H])n1cc(S(=O)(=O)NC2CCNCC2(F)F)c(Cl)c1C(=O)Nc1cc(F)c(F)c(F)c1 ; [2H]C([2H])n1cc(S(=O)(=O)NC2CCNCC2(F)F)c(Cl)c1C(=O)Nc1cc(F)c(F)c(F)c1 ; [2H]Cn1cc(S(=O)(=O)NC2CCNCC2(F)F)c(Cl)c1C(=O)Nc1cc(F)c(F)c(F)c1]

The compounds provided herein are intended to encompass all possible stereoisomers, unless a particular stereochemistry is specified. Where the compound provided herein contains an alkenyl or alkenylene group, the compound may exist as one or a mixture of geometric cis/trans (or Z/E) isomers. Where structural isomers are interconvertible, the compound may exist as a single tautomer or a mixture of tautomers. This can take the form of proton tautomerism in the compound that contains, for example, an imino, keto, or oxime group; or so-called valence tautomerism in the compound that contain an aromatic moiety. It follows that a single compound may exhibit more than one type of isomerism.

The compounds provided herein may be enantiomerically pure, such as a single enantiomer or a single diastereomer, or be stereoisomeric mixtures, such as mixture of enantiomers, e.g., a racemic mixture of two enantiomers; or a mixture of two or more diastereomers. As such, one of skill in the art will recognize that administration of a compound in its (R) form is equivalent, for compounds that undergo epimerization in vivo, to administration of the compound in its (S) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

When the compound provided herein contains an acidic or basic moiety, it may also be provided as a pharmaceutically acceptable salt. The pharmaceutically acceptable salts are generally prepared from pharmaceutically acceptable non-toxic bases or acids including inorganic or organic bases and inorganic or organic acids. Suitable acids for use in the preparation of pharmaceutically acceptable salts include, but are not limited to, acetate, ascorbate, adipate, alginate, aspirate, benzenesulfonate, benzoate, bicarbonate, bisulfate, bitartrate, borate, bromide, butyrate, camphorate, camphorsulfonate, camsylate, carbonate, chloride, clavulanate, citrate, cyclopentane propionate, diethylacetic, digluconate, dihydrochloride, dodecylsulfanate, edetate, edisylate, estolate, esylate, ethanesulfonate, formic, fumarate, gluceptate, glucoheptanoate, gluconate, glutamate, glycerophosphate, glycollylarsanilate, hemisulfate, heptanoate, hexanoate, hexylresorcinate, hydrabamine, hydrobromide, hydrochloride, 2-hydroxyethanesulfonate, hydroxynaphthoate, iodide, isonicotinic, isothionate, lactate, lactobionate, laurate, malate, maleate, mandelate, mesylate, methylbromide, methylnitrate, methylsulfate, methanesulfonate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, phosphate/diphosphate, pimelic, phenylpropionic, polygalacturonate, propionate, salicylate, stearate, sulfate, subacetate, succinate, tannate, tartrate, teoclate, thiocyanate, tosylate, triethiodide, trifluoroacetate, undeconate, valerate and the like. Suitable bases for use in the preparation of pharmaceutically acceptable salts include, but are not limited to, magnesium hydroxide, calcium hydroxide, potassium hydroxide, zinc hydroxide, sodium hydroxide, primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, tromethamine and the like.

The compound provided herein may also be provided as a prodrug, which is a functional derivative of the compound, for example, of Formula (I), and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis.

Provided herein are pharmaceutical compositions comprising a compound provided herein, e.g., a compound of Formula (I), as an active ingredient, including a stereoisomer, or diastereomer thereof; or a pharmaceutically acceptable salt, solvate, or prodrug thereof; and one or more pharmaceutically acceptable carriers or excipients.

Suitable carriers or excipients are well known to those skilled in the art, and non-limiting examples of suitable excipients are provided herein. Whether a particular excipient is suitable for incorporation into a pharmaceutical composition or dosage form depends on a variety of factors well known in the art, including, but not limited to, the method of administration. For example, oral dosage forms such as tablets may contain excipients not suited for use in parenteral dosage forms. The suitability of a particular excipient may also depend on the specific active ingredients in the dosage form.

The pharmaceutical compositions of the present disclosure comprise a compound provided herein (e.g., a compound of Formula (I), including a stereoisomer, or diastereomer thereof; or a pharmaceutically acceptable salt, solvate, or prodrug thereof) as an active ingredient, one or more pharmaceutically acceptable carriers/excipients and optionally other therapeutic ingredients or adjuvants. The compositions include compositions suitable for oral, rectal, topical, and parenteral (including subcutaneous, intramuscular, and intravenous) administration. These dosage forms can be prepared according to conventional methods and techniques known to those skilled in the art.

The pharmaceutical compositions provided herein can be provided in a unit-dosage form or multiple-dosage form. A unit-dosage form, as used herein, refers to physically discrete a unit suitable for administration to a human and animal subject, and packaged individually as is known in the art. Each unit-dose contains a predetermined quantity of an active ingredient(s) sufficient to produce the desired therapeutic effect, in association with the required pharmaceutical carriers or excipients. Examples of a unit-dosage form include an ampoule, syringe, and individually packaged tablet and capsule. For example, a 100 mg unit dose contains about 100 mg of an active ingredient in a packaged tablet or capsule. A unit-dosage form may be administered in fractions or multiples thereof. A multiple-dosage form is a plurality of identical unit-dosage forms packaged in a single container to be administered in segregated unit-dosage form. Examples of a multiple-dosage form include a vial, bottle of tablets or capsules, or bottle of pints or gallons.

The pharmaceutical compositions provided herein can be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

The pharmaceutical compositions provided herein for oral administration can be provided in solid, semisolid, or liquid dosage forms for oral administration. As used herein, oral administration also includes buccal, lingual, and sublingual administration. Suitable oral dosage forms include, but are not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, syrups, lipsomes, micelles, microspheres, nanosystems, sustained release formulations, and the like. In addition to the active ingredient(s), the pharmaceutical compositions can contain one or more pharmaceutically acceptable carriers or excipients, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide.

Binders or granulators impart cohesiveness to a tablet to ensure the tablet remaining intact after compression. Suitable binders or granulators include, but are not limited to, starches; gelatin; sugars; natural and synthetic gums, such as acacia, alginic acid, alginates, carboxymethylcellulose, methylcellulose, polyvinylpyrrolidone (PVP); celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses; and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol.

Suitable disintegrants include, but are not limited to, agar; bentonite; celluloses; wood products; natural sponge; cation-exchange resins; alginic acid; gums; citrus pulp; cross-linked celluloses, such as croscarmellose; cross-linked polymers, such as crospovidone; cross-linked starches; calcium carbonate; microcrystalline cellulose; starches; clays; aligns; and mixtures thereof.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels; and mixtures thereof.

Suitable glidants include, but are not limited to, colloidal silicon dioxide and asbestos-free talc. Suitable coloring agents include, but are not limited to, any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Suitable flavoring agents include, but are not limited to, natural flavors extracted from plants, such as fruits, and synthetic blends of compounds which produce a pleasant taste sensation, such as peppermint and methyl salicylate. Suitable sweetening agents include, but are not limited to, sucrose, lactose, mannitol, syrups, glycerin, and artificial sweeteners, such as saccharin and aspartame. Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suitable suspending and dispersing agents include, but are not limited to, sodium carboxymethylcellulose, pectin, tragacanth, Veegum, acacia, sodium carbomethylcellulose, hydroxypropyl methylcellulose, and polyvinylpyrrolidone. Suitable preservatives include, but are not limited to, glycerin, methyl and propylparaben, benzoic add, sodium benzoate and alcohol. Suitable wetting agents include, but are not limited to, propylene glycol monostearate, sorbitan monooleate, diethylene glycol monolaurate, and polyoxyethylene lauryl ether. Suitable solvents include, but are not limited to, glycerin, sorbitol, ethyl alcohol, and syrup. Suitable non-aqueous liquids utilized in emulsions include, but are not limited to, mineral oil and cottonseed oil. Suitable organic acids include, but are not limited to, citric and tartaric acid. Suitable sources of carbon dioxide include, but are not limited to, sodium bicarbonate and sodium carbonate.

The pharmaceutical compositions provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, intravesical, and subcutaneous administration. The pharmaceutical compositions provided herein for parenteral administration can be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science.

The pharmaceutical compositions intended for parenteral administration can include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases. Suitable aqueous vehicles include, but are not limited to, water, saline, physiological saline or phosphate buffered saline (PB S), sodium chloride injection, Ringers injection, isotonic dextrose injection, sterile water injection, dextrose and lactated Ringers injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Suitable water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

The pharmaceutical compositions provided herein can be administered topically to the skin, orifices, or mucosa. The topical administration, as used herein, includes (intra)dermal, conjunctival, intracorneal, intraocular, ophthalmic, auricular, transdermal, nasal, vaginal, urethral, respiratory, and rectal administration. The pharmaceutical compositions provided herein can be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, and dermal patches. The topical formulation of the pharmaceutical compositions provided herein can also comprise liposomes, micelles, microspheres, nanosystems, and mixtures thereof. Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

Also provided herein is a combination of a compound disclosed herein, e.g., a compound of Formula (I), including a stereoisomer, or enantiomer thereof; or a pharmaceutically acceptable salt, stereoisomer, solvate, or prodrug thereof with one or more additional therapeutic agents (such as, but not limited to a second and different anti-HBV infection agent). For instance, the compounds of this disclosure can be combined with one or more anti-HBV agents such as reverse transcriptase inhibitors, capsid inhibitors, cccDNA formation inhibitors, HbsAg release inhibitors, oligomeric nucleotides targeted to the Hepatitis B genome, or immunostimulators. In one embodiment, the compounds of this disclosure are also useful in combination with one or more additional therapeutic agents for simultaneous, separate or sequential administration.

In one embodiment, the reverse transcriptase inhibitors include, but are not limited to entecavir, clevudine, telbivudine, lamivudine, adefovir, tenofovir, adefovir dipovoxil, emtricitabine, abaccavir, elvucitabine, ganciclovir, lobucavir, famciclovir, penciclovir, amdoxovirl, and the like.

In one embodiment, the capsid inhibitors may include, but is not limited to, any compound that inhibits capsid assembly, induces formation of non-capsid polymers, promotes excess capsid assembly or misdirected capsid assembly, affects capsid stabilization, or inhibits encapsidation of RNA (pgRNA).

In one embodiment, the cccDNA formation inhibitors include compounds that are capable of inhibiting the formation and/or stability of cccDNA either directly or indirectly. For example, a cccDNA formation inhibitor may include, but is not limited to, any compound that inhibits capsid disassembly, rcDNA entry into the nucleus, or the conversion of rcDNA into cccDNA.

In one embodiment, HBsAg release inhibitors include compounds that are capable of inhibiting, either directly or indirectly, the secretion of sAg (S, M and/or L surface antigens) bearing subviral particles and/or DNA containing viral particles from HBV-infected cells.

In one embodiment, oligomeric nucleotides targeted to the Hepatitis B genome include, but are not limited to, isolated, double stranded, siRNA molecules, that each include a sense strand and an antisense strand that is hybridized to the sense strand, such as Arrowhead-ARC-520.

In one embodiment, immunostimulators include, but are not limited to, agonists of stimulator of IFN genes (STING) and interleukins, interferons, TLR-7 agonists (such as, but not limited to, GS-9620, RG-7795), T-cell stimulators (such as, but not limited to, GS-4774), RIG-1 inhibitors (such as, but not limited to, SB-9200), or SMAC-mimetics (such as, but not limited to, Birinapant).

Also provided herein a kit comprising a compound disclosed herein, e.g., a compound of Formula (I) or (II), including a stereoisomer, or enantiomer thereof; or a pharmaceutically acceptable salt, stereoisomer, solvate, or prodrug thereof. The kit may further comprise instructions for use, e.g., for use in treating a HBV infection. The instructions for use are generally written instructions, although electronic storage media (e.g., magnetic diskette or optical disk) containing instructions are also acceptable.

Described below are the procedures used to synthesize the exemplary compounds.

All the reagents and solvents were purchased from commercial sources and used without further purification unless otherwise indication. All the reactions were carried out under dry nitrogen or argon atmosphere and monitored by thin layer chromatography (TLC) using Merck Silica Gel 60 F₂₅₄ glass-backed plate. Column chromatography was performed by Merck Silica Gel 60 (0.040-0.063 mm, 230-400 mesh). ¹H NMR and ¹³C NMR spectra were measured by Varian Mercury-300 and Varian Bruker AVIII-500 spectrometers, and the chemical shifts (δ) were reported in parts per million (ppm) relative to the resonance of the solvent peak. Multiplicities are reported with the following abbreviations: s (singlet), d (doublet), t (triplet), q (quartet), quin (quinete), m (multiplet), or br (broad). Low-resolution mass spectra were measured by HP Hewlett Packard 1100 series.

The following scheme I was followed for synthesizing the compounds of Formula (I):

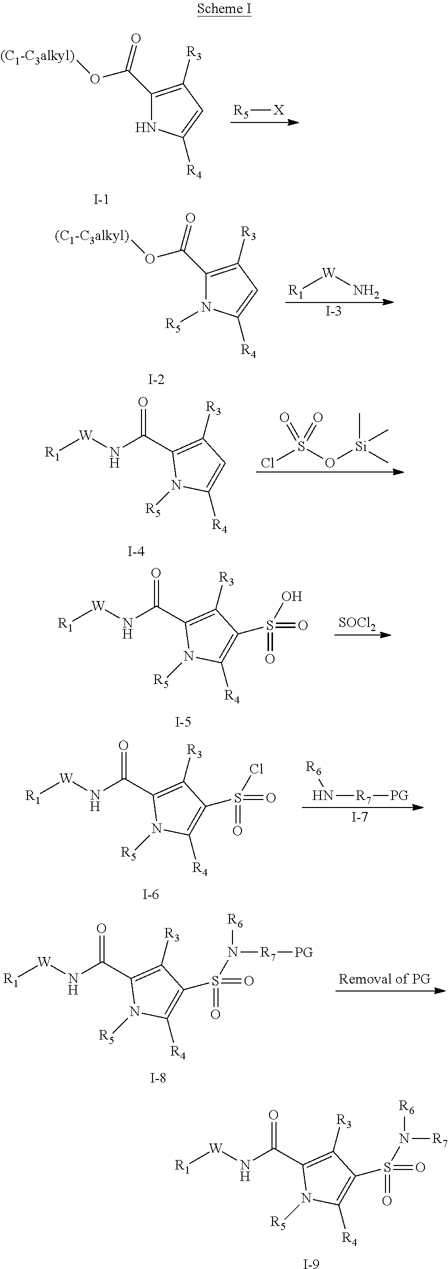

A compound of Formula (I) can be prepared as shown in Scheme 1. Compound I-1 is first converted to compound I-2 by reacting with R₅—X. Compound I-2 then reacts with compound I-3 to form compound I-4. Subsequently, compound I-4 reacts with trimethylsilyl chlorosulfonate to form compound I-5, which is then treated with SOCl₂ to form compound I-6. Compound I-6 is converted to compound I-8 by reacting with compound I-7. Compound I-8 is converted to compound I-9 by removing the PG (protecting group). For the compounds shown in Scheme 1, wherein R₁, R₃, R₄, R₅, R₆, R₇ and W are defined as in any of the embodiments described herein.

The disclosure will be further understood by the following non-limiting example.

### EXAMPLE 1

### Preparation and Characterization of Compound 1-3

For all of the following examples, standard work-up and purification methods known to those skilled in the art can be utilized. Unless otherwise indicated, all temperatures are expressed in ° C. (degrees Centigrade). All reactions conducted at room temperature unless otherwise noted. Synthetic methodologies herein are intended to exemplify the applicable chemistry through the use of specific examples and are not indicative of the scope of the disclosure.

The starting materials used in the examples described herein are either commercially available or can be prepared by a method known to one of skill in the art.

### Synthesis and Characterization of Compound 1

3-Chloro-4-(3,3-difluoro-piperidin-4-ylsulfamoyl)-1-methyl-d₃-1H-pyrrole-2-carboxylic acid (3,4,5-trifluoro-phenyl)-amide (Compound 1)

Compound 1-ii was first prepared from commercially available 3-Chloro-1H-pyrrole-2-carboxylic acid methyl ester via intermediate 1-i, following the scheme shown below:

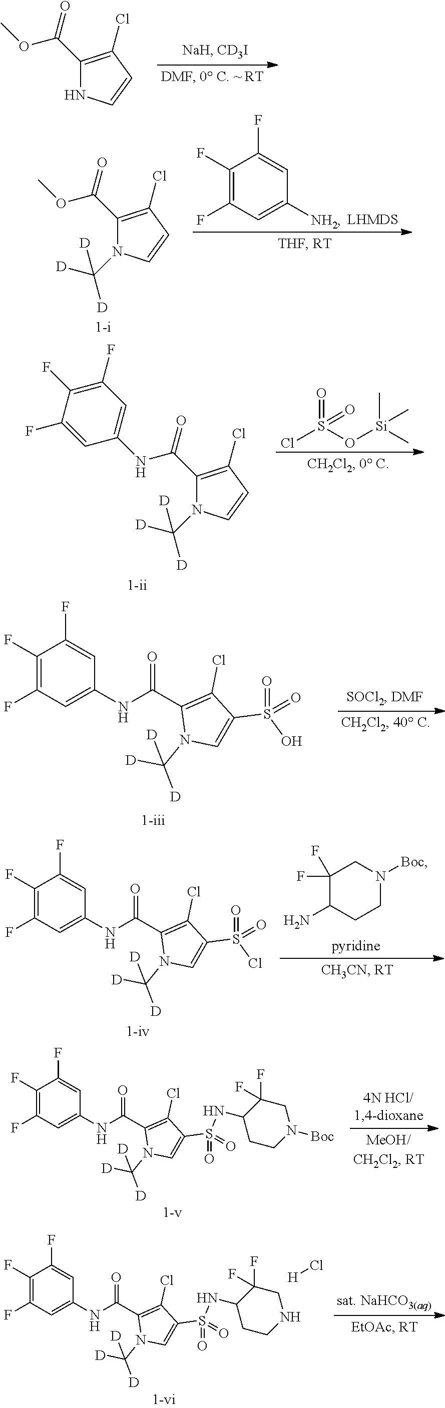

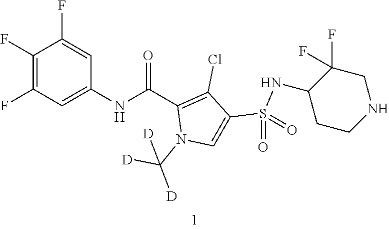 [SMILES: [2H]C([2H])([2H])n1cc(S(=O)(=O)NC2CCNCC2(F)F)c(Cl)c1C(=O)Nc1cc(F)c(F)c(F)c1]

To a solution of Methyl 3-chloro-1H-pyrrole-2-carboxylate (30 g, 191 mmole) in dry DMF (600 mL) was added Sodium hydride (9.16 mg, 229 mmole, 60% in mineral oil) at 0° C., the mixture was stirred for 30 min, then Methyl-d₃ iodide (18 mL, 289 mmole) was added dropwise at ice bath over 15 min, the reaction mixture was allowed to warm to room temperature and stirred for 16 hours. The reaction mixture was acidified with 1 N HCl_{(aq)} and concentrated. The residue was dissolved in H₂O/EtOAc. The organic layer was dried over MgSO₄, filtered and concentrated to obtain crude intermediate 1-i as a light yellow oil, which was used directly for next step without further purification.

To a solution of compound 1-i in dry THF (500 mL) was added 3,4,5-Trifluoroaniline (33.7 g, 229 mmole) at room temperature, the mixture was stirred for 10 min. Then Lithium bis(trimethylsilyl)amide solution (306 mL, 306 mmole, 1.0 M in THF) was added dropwise at ice-water bath. After the addition finished, the reaction mixture was allowed to stir at room temperature until reaction was completed by checking with LC-MS. Then the mixture was diluted with EtOAc, washed with 1 N HCl_{(aq)}, H₂O and brine sequentially. The organic layer was dried over MgSO₄, filtered and concentrated. The obtained residue was purified by column chromatography on silica gel eluting with EtOAc/Hexanes (1:9) to give compound 1-ii as a light yellow solid (31.7 g, yield=57% in two steps).

To a solution of compound 1-ii (6.0 g, 20.6 mmole) in dry CH₂Cl₂ (200 mL) was added Trimethylsilyl chlorosulfonate (5.8 g, 30.7 mmole) at 0° C., the mixture was stirred under ice-water bath for 1 hour to afford compound 1-iii as a light yellow solid, which was suspension in CH₂Cl₂. After reaction completed, SOCl₂ (15.0 mL, 206 mmole) and dry DMF (3.2 mL, 41.3 mmole) were added to the above solution, kept the solution clear and heated to 40° C. for 2 hours. The reaction mixture was concentrated, and the obtained dark yellow oil was subjected to silica gel column chromatography using EtOAc as eluant, the product fractions were concentrated and recrystalized in CH₂Cl₂/Hexanes to give compound 1-iv as an off-white solid (5.8 g, yield =75% in two steps).

Compound 1 was prepared via intermediates 1-iv to 1-vi as follows. A solution of compound 1-iv (1.5 g, 4.0 mmole) in CH₃CN (20 mL) was added 4-Amino-3,3-difluoro-piperidine-1-carboxylic acid tert-butyl ester (1.0 g, 4.2 mmole) and Pyridine (1.0 mL, 12.4 mmole) at ice-water bath, the reaction mixture was allowed to warm to room temperature and stirred for 3 hours. Quenched the reaction with 1 N HCl_{(aq)}, washed the mixture with H₂O and brine. The organic layer was dried over MgSO₄, filtered and concentrated. The obtained residue was purified by column chromatography on silica gel using a gradient from 10 to 50% EtOAc in Hexanes to afford compound 1-v as an off-white solid (2.2 g, yield=93%).

The compound 1-v (2.2 g, 3.7 mmole) was dissolved in CH₂Cl₂/MeOH (1:1, 22 mL) was added 4 N HCl/1,4-dioxane (4.6 mL, 18.4 mmole), and the mixture was stirred at room temperature for 3 hours, evaporated to give the deprotected compound 1-vi as a light yellow hydrochloride salt (quantitative yield).

To a solution of compound 1-vi in H₂O (20 mL) was neutralized with sat. NaHCO_{3 (aq)} at ice-water bath, then the aqueous solution was extracted with EtOAc (50 mL×3). The combined organic layers were dried over MgSO₄, filtered and concentrated. The crude product was recrystalized in CH₂Cl₂/Hexanes system to afford compound 1 as an off-white solid (1.5 g, yield=83% in two steps).

Compound 2 and Compound 3 are shown in the table below. Each of Compound 2 and 3 was similarly prepared following the scheme as set forth above and the protocols described in the pre

Each of Compounds 2-3 was similarly prepared following the scheme as set forth above and the protocols described in the Synthesis and Characterization of Compound 1.

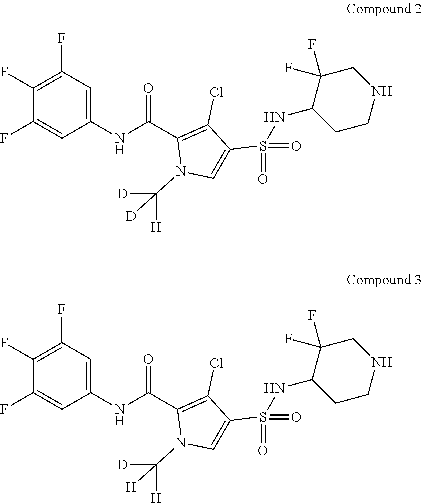 [SMILES: [2H]C([2H])n1cc(S(=O)(=O)NC2CCNCC2(F)F)c(Cl)c1C(=O)Nc1cc(F)c(F)c(F)c1 ; [2H]Cn1cc(S(=O)(=O)NC2CCNCC2(F)F)c(Cl)c1C(=O)Nc1cc(F)c(F)c(F)c1]

### EXAMPLE 2

### Real-Time PCR Assay

HepAD38 cells were seeded and cultured on 96-well plates. After incubation for 2 days, cells were fed with compound-containing media without tetracycline. After compound treatment for 5 days, culture supernatants were collected. Extracellular DNA was then extracted using LabTurbo DNA mini kit, and quantified by real-time PCR.

Real-time PCR was performed using an ABI QuantStudio 3 system in a 96-well optical plate format. The PCR mixture containing forward primer (5′-ACATCAGGATTCCTAGGACC-3′), reverse primer (5′-GGTGAGTGATTGGAGGTTG-3′) and Luna Universal qPCR Master Mix in a final volume of 24 μl was incubated at 95° C. for 10 min followed by 45 cycle of incubation at 95° C. for 10 s and 60° C. for 10 s. Regression analysis was performed using GraphPad Prism 5 to calculate the 50% effective concentration (EC₅₀) values. The compounds of Formula (I) exhibited high potency in inhibiting the HBV replication.

### EXAMPLE 3

### In-Vivo Pharmacokinetic Study

Animals were adapted to laboratory conditions for at least 1 week prior to dosing and were allowed food and water ad libitum. The actual volume administered was based on the body weight of each animal before dosing. All animals were euthanized by CO₂ inhalation before the time point of blood collection. About 0.4 ml blood was collected from each animal by cardiac puncture at designated time point. Sampling time points were at 0.25, 0.5, 1, 2, 4, 6, 8, 16, and 24 hrs post-dosing. Blood samples were collected into heparin containing tubes and placed on crushed wet ice immediately after collection. Blood samples were then centrifuged at 12,000 rpm for 5 minutes at 4° C. to separate the cellular and plasma components. The plasma samples were collected and stored at −80° C. until analysis. Liver tissues were collected and chilled on crushed wet ice. Liver tissues were then weighed, transferred, and stored at −80° C. until analysis. Plasma were analyzed using LC-MS/MS method. The tissues were also analyzed using LC-MS/MS method if necessary. Individual plasma and tissue concentrations at designated sampling time points were calculated using Analyst™ Software. Pharmacokinetic parameters were performed using WinNonlin® pharmacokinetic software (Version 4.0, Statistical Consultants Inc., Kentucky, USA). A non-compartmental approach consistent with the oral route of administration was used for PK parameters estimation. Nominal dose and protocol designated sampling time points were used in calculation. The following parameters were estimated: the plasma elimination half-time (t_{1/2}; 0.693/λ), Cₘₐₓ, Tₘₐₓ, area under the concentration-time curve from hour 0 to last sampling time point (AUC₀₋ₜ) and area under the concentration-time curve extrapolated to infinity (AUC_{0-inf}).

### Other Embodiments

All of the features disclosed in this specification may be combined in any combination. Each feature disclosed in this specification may be replaced by an alternative feature serving the same, equivalent or similar purpose. Thus, unless expressly stated otherwise, each feature disclosed is only an example of a series of equivalent or similar features.

From the above description, one skilled in the art can easily ascertain the essential characteristics of the present disclosure, and without departing from the spirit and scope thereof, can make various changes and modifications of the disclosure to adapt it to various usage and conditions. Thus, other embodiments are also within the scope of the following claims.

What is claimed is:

1. A compound of Formula (I) below, or a pharmaceutically acceptable salt, stereoisomer, enantiomer, solvate, or prodrug thereof, cm 2. 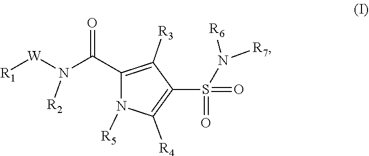
wherein
R₁ is C₁₋₆ alkyl, C₂₋₆ alkenyl, C₁₋₆ alkoxy, C₃₋₁₂ carbocyclyl, C₃₋₁₂ heterocyclyl, aryl, or C₅₋₁₀ heteroaryl, wherein said C₁₋₆ alkyl, C₂₋₆ alkenyl, C₁₋₆ alkoxy, C₃₋₁₂ carbocyclyl, C₃₋₁₂ heterocyclyl, aryl, and C₅₋₁₀ heteroaryl is each independently optionally substituted with 1 to 4 substituents each independently selected from hydrogen, halogen, NH₂, OH, CN, C₁₋₆ alkyl, C₂₋₆ alkenyl, C₁₋₆ alkoxy, C₁₋₆ haloalkyl, C₁₋₆ haloalkoxy, C₃₋₆ carbocyclyl, C₃₋₆ heterocyclyl, aryl, and C₅₋₆ heteroaryl;
W is a bond, C₁₋₆ alkyl, or C₂₋₆ alkenyl;
R₂ is hydrogen, C₁₋₆ alkyl, or C₁₋₆ haloalkyl;
R₃ is hydrogen, halogen, CN, C₁₋₆ alkyl or C₁₋₆ haloalkyl;
R₄ is hydrogen, halogen, CN, C₁₋₆ alkyl or C₁₋₆ haloalkyl;
R₅ is C₁₋₆ alkyl optionally substituted with 1 to 3 deuteriums;
R₆ is hydrogen, C₁₋₆ alkyl, or C₁₋₆ haloalkyl;
R₇ is C₃₋₁₂ heterocyclyl, aryl, C₅₋₁₀ heteroaryl, C₁₋₆ alkyl-C₃₋₁₂ heterocyclyl, C₁₋₆ alkyl-aryl, or C₁₋₆ alkyl-C₅₋₁₀ heteroaryl wherein said C₃₋₁₂ heterocyclyl, aryl, C₅ heteroaryl, C₁₋₆ alkyl-C₃₋₁₂ heterocyclyl, C₁₋₆ alkyl-aryl, or C₁₋₆ alkyl-C₅₋₁₀ heteroaryl is each independently optionally substituted with one or more substituents each independently selected from hydrogen, halogen, OH, CN, C₁₋₆ alkyl, C₂₋₆ alkenyl, C₁₋₆ alkoxy, C₁₋₆ haloalkyl, C₃₋₁₂ heterocyclyl, phosphate ester, C₁₋₆ alkyl-R₈, C₂₋₆ alkenyl-R₈, C₁₋₆ alkoxy-R₈, —NHS(O)₂—R₈, —S(O)₂—N(R₈)₂, —NHS(O)₂—N(R₈)₂, NH—C(═O)—N(R₈)₂, —C(═O)—C(═O)—R₈, NH—C(═O)—C(═O)—R₈, —C(═O)—C(═O)—N(R₈)₂, or NH—C(═O)—C(═O)—N(R₈)₂, wherein said C₁₋₆ alkyl, C₂₋₆ alkenyl, C₁₋₆ alkoxy, C₁₋₆ haloalkyl, C₃₋₁₂ heterocyclyl, phosphate ester, C₁₋₆ alkyl-R₈, C₂₋₆ alkenyl-R₈, C₁₋₆ alkoxy-R₈, —NHS(O)₂—R₈, —S(O)₂—N(R₈)₂, —NHS(O)₂—N(R₈)₂, NH—C(═O)—N(R₈)₂, —C(═O)—C(═O)—R₈, NH—C(═O)—C(═O)—R₈, —C(═O)—C(═O)—N(R₈)₂, or NH—C(═O)—C(═O)—N(R₈)₂ is each independently optionally substituted with one or more substituents each independently selected from hydrogen, halogen, OH, CN, oxo, C₁₋₆ alkyl, or C₁₋₆ haloalkyl;
R₈ is hydrogen, halogen, OH, CN, C₁₋₆ alkyl, C₂₋₆ alkenyl, C₁₋₆ alkoxy, C₃₋₁₂ carbocyclyl, C₃₋₁₂ heterocyclyl, aryl, or C₅₋₁₄ heteroaryl, wherein said C₁₋₆ alkyl, C₂₋₆ alkenyl, C₁₋₆ alkoxy, C₃₋₁₂ carbocyclyl, C₃₋₁₂ heterocyclyl, aryl, or C₅₋₁₄ heteroaryl is each independently optionally substituted with one or more halogen, OH, CN, C₁₋₆ alkyl, C₂₋₆ alkenyl, C₁₋₆ haloalkyl, or C₁₋₆ alkoxy.
